(19) United States
(12) Patent Application Publication
(10) Pub. No.: US 2020/0054268 A1
(43) Pub. Date: Feb. 20, 2020

(54) METHOD OF GENERATION OF A DIAGNOSTIC INDEX FOR ALZHEIMER DISEASE, ELECTRONIC APPARATUS FOR IMPLEMENTATION OF THE METHOD AND SYSTEM

(71) Applicant: UNIVERSITA' DEGLI STUDI DI BRESCIA, Brescia (IT)

(72) Inventors: Alessandro PADOVANI, Brescia (IT); Barbara BORRONI, Brescia (IT); Alberto BENUSSI, Brescia (IT); Alessandro DEPARI, Brescia (IT)

(21) Appl. No.: 16/346,748

(22) PCT Filed: Oct. 30, 2017
(86) PCT No.: PCT/IB2017/056715

§ 371 Date: May 1, 2019

(30) Foreign Application Priority Data
Nov. 2, 2016 (IT) 102016000110051

(51) Int. Cl. A61B 5/00; A61B 5/0488

(52) U.S. Cl. CPC A61B 5/4088; A61N 2/006; A61B 5/6825; A61B 5/0488

(57) ABSTRACT

A method of generation of diagnostic index for Alzheimer's disease provides for stimulating a subject using transcranial magnetic stimulation with a conditioning stimulus and, after a first predetermined time interval, with control stimulus and to record amplitude of first motor evoked potential generated as a result of the conditioning and control stimuli. A second conditioning stimulus is administered after a second predetermined time interval greater than the first predetermined time interval, allowing recording amplitude of second motor evoked potential. A third conditioning stimulus is administered using nervous electrical stimulation after a third predetermined time interval, allowing recording amplitude of third motor evoked potential generated as a result of the third conditioning stimulus. Subsequently, the method provides for generating diagnostic index on a basis of processing of the first, second and third recorded motor evoked potential.

The object of the present invention is a method for generating a diagnostic index to facilitate the early diagnosis of Alzheimer's disease; in particular, the object of the present invention is also an electronic apparatus and a system for implementing the method.

The early diagnosis of Alzheimer's disease requires reliable and trustworthy diagnostic markers. The scientific studies so far performed have identified biological markers of accumulation or of neuronal damage, through the dosage of cerebrospinal fluid proteins (e.g. Tau, beta-amyloid) or through cerebral imaging methods (e.g. PET with amyloid tracer or quantification of the cerebral atrophy with nuclear magnetic resonance).

However, the early diagnosis of the disease is currently difficult due to the absence of reliable and trustworthy biological markers. In addition, the differential diagnosis with other non-Alzheimer dementia-syndrome neurodegenerative pathologies often provides for the biomarker acquisition through the use of invasive or expensive methods, such as spinal tap or the administration of radiolabeled tracers, procedures that moreover are not always feasible in all centres, as they are not available.

One of the objects of the present invention is to provide a method of generating a diagnostic index suitable to facilitate the early diagnosis of the disease, overcoming the above drawbacks of the prior art. In particular, one of the objects of the present invention is to propose a less invasive method with respect to the above methods of the prior art and at the same time capable of facilitating both an early and differential diagnosis of the disease, making it feasible in most centres intended for diagnostic testing.

Such an object is achieved by a method of generating a diagnosis index, by an electronic apparatus and by a stimulation management system according to the accompanying independent claims; the dependent claims describe preferred embodiment variants.

The features and advantages of the method, of the apparatus and of the system according to the present invention will appear more clearly from the following description, made by way of an indicative and non-limiting example, with reference to the accompanying tables, in which:

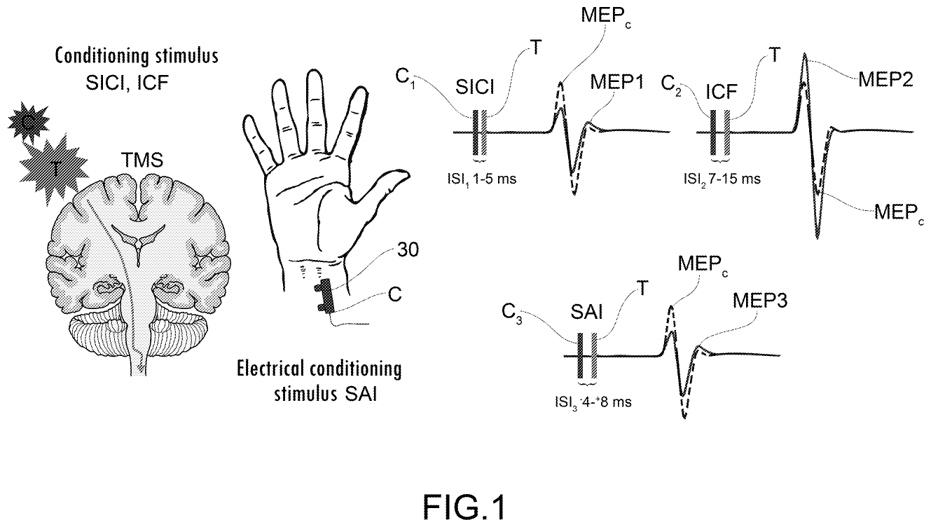

FIG. 1 schematically shows three different stimulation protocols for evaluating short-interval intracortical inhibition (SICI), intracortical facilitation (ICF) and short-latency afferent inhibition (SAI);

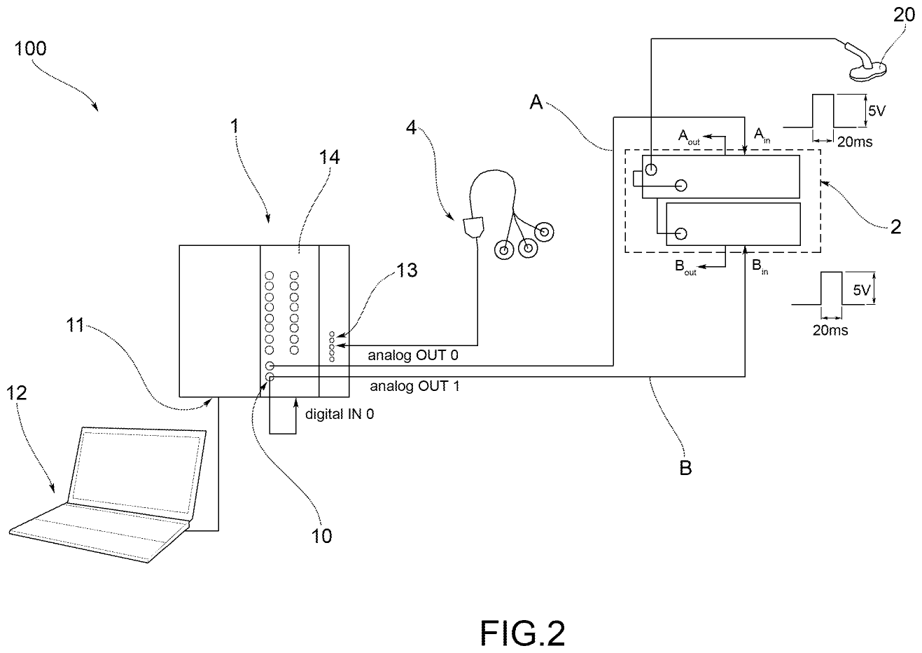

FIG. 2 schematically shows a stimulation management system 100, comprising an electronic apparatus 1 according to an embodiment variant of the present invention;

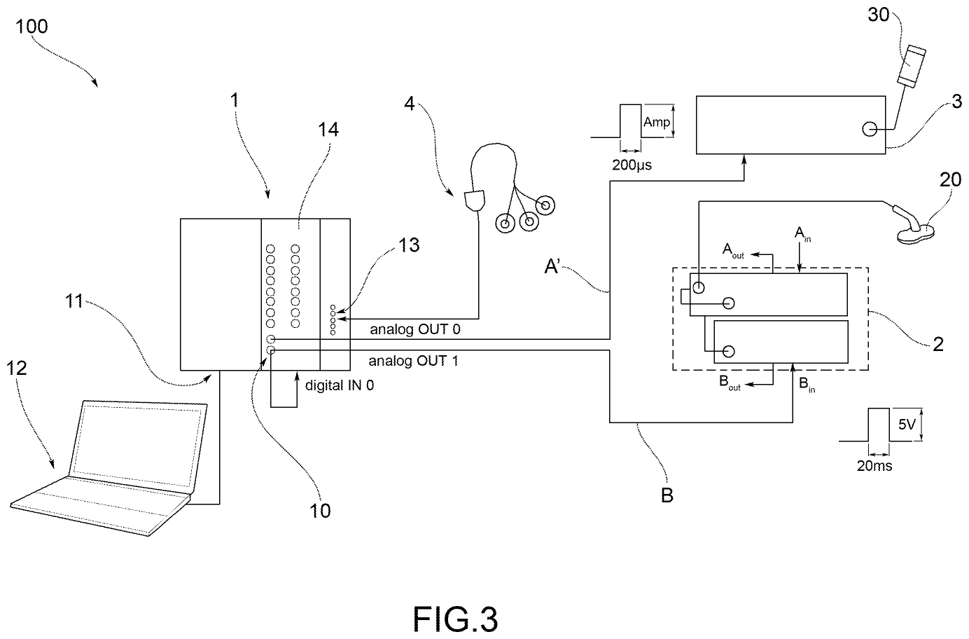

FIG. 3 schematically shows a stimulation management system 100, comprising an electronic apparatus 1 according to said embodiment variant shown in FIG. 2, but in another configuration;

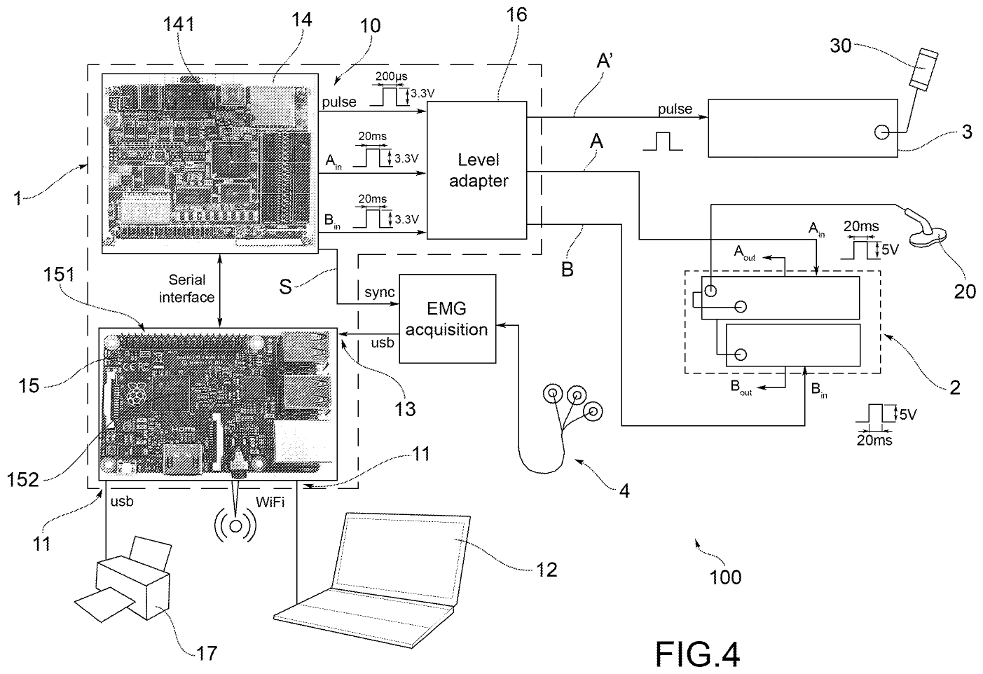

FIG. 4 schematically shows a stimulation management system 100, comprising an electronic apparatus 1 according to another embodiment variant of the present invention, wherein the electronic apparatus is an integrated device;

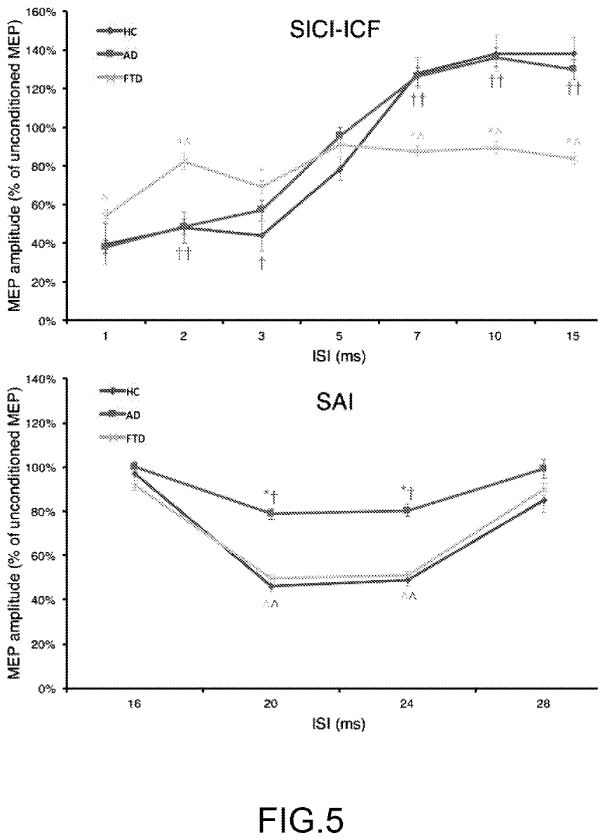

FIG. 5 shows two graphs representing the results of a clinical study described below in the text, wherein one of the two graphs entitled ISI-ICF shows the data relative to the averages (with standard error represented by bars) of the ratios between the amplitudes of the detected motor evoked potentials for each paradigm (SICI and ICF) at each ISI and the amplitude of the average of the control motor evoked potentials, and wherein the other graph, entitled SAI, shows the data relative to the averages (with standard error represented by bars) of the ratios between the amplitudes of the detected motor evoked potentials for the SAI paradigm at each ISI and the amplitude of the average of the control motor evoked potentials; said averages for the paradigms SICI, ICF and SAI are shown for all subject groups of the study: healthy (HC), with Alzheimer's disease (AD) and with frontotemporal disease (FTD);

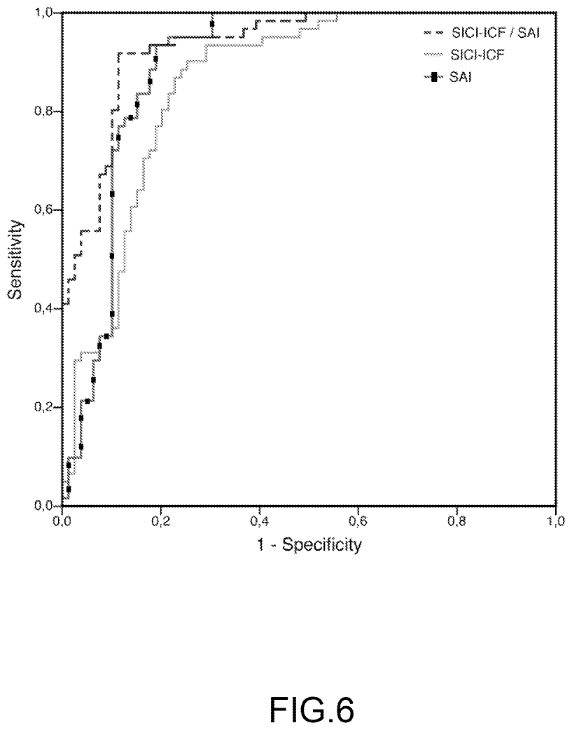

FIG. 6 shows the ROC curves for the neurophysiological parameters in SICI, ICF, SAI and ratio thereof in the differentiation of Alzheimer's disease with respect to the frontotemporal disease.

Transcranial magnetic stimulation is a non-invasive technique based on the electromagnetic induction principle, which provides for the administration of a magnetic impulse through a coil on the scalp of a subject that inducts a transitory electric current in the underlying cerebral surface, which determines the selective activation of predetermined cortical neuron populations. Preferably, the coil is positioned on the area of the scalp above the left motor cortex (as shown for example in FIG. 1), with the handle of the coil facing downwards and laterally at 45° with respect to the median line of the cranium. Such a position allows evoking a motor evoked potential (MEP) that is preferably recorded as an electric signal at the level of the first dorsal interosseous muscle (FDI) of the right hand through an electromyographic signal detection or acquisition device.

According to the invention, the method of generating a diagnostic index to facilitate the diagnosis of Alzheimer's disease uses the above non-invasive transcranial magnetic stimulation technique, per se known in the field for investigating other types of pathologies.

With reference to the accompanying figures, the method according to the invention comprises the following steps:

a1) stimulating a subject using transcranial magnetic stimulation with at least one first conditioning stimulus (indicated by C1 in FIG. 1) and, after a first predetermined time interval (indicated by ISI1, inter-stimulus interval, in FIG. 1), further stimulating the subject using transcranial magnetic stimulation with a control stimulus (indicated by T in FIG. 1) and recording the amplitude of a first motor evoked potential (indicated by MEP1 in FIG. 1) generated by a region of the body of the subject, for example near the first dorsal interosseous muscle, as a result of said conditioning and control stimuli;
b1) generating a first comparison index by comparing said amplitude of the first motor evoked potential MEP1 and the amplitude of a control motor evoked potential (indicated by MEPc in FIG. 1);
a2) stimulating a subject using transcranial magnetic stimulation with at least a second conditioning stimulus C2 and, after a second time interval ISI2 greater than such a first predetermined time interval ISI1, further stimulating the subject using transcranial magnetic stimulation with a control stimulus T and recording the amplitude of a second motor evoked potential MEP2 generated by the region of the body of the subject as a result of said second conditioning C2 and control stimuli T;
b2) generating a second comparison index by comparing the amplitude of the second motor evoked potential MEP2 and the amplitude of the control motor evoked potential MEPc;
a3) stimulating a subject using nervous electrical stimulation of a nerve associated with said region of the body with at least one electrical conditioning stimulus C3 and, after a third predetermined time interval ISI3, further stimulating the subject using transcranial magnetic stimulation with a control stimulus T and recording the amplitude of a third motor evoked potential MEP3 generated by the region of the body of the subject as a result of said third conditioning C3 and control stimuli T;
b3) generating a third comparison index by comparing said amplitude of the third motor evoked potential MEP3 and the amplitude of the control motor evoked potential MEPc;
c) elaborating said first comparison index, second comparison index and third comparison index to obtain a diagnostic comparison index as a function of said first comparison index, second comparison index and third comparison index.

Preferably, the diagnostic comparison index is a ratio between said first comparison index, second comparison index and third comparison index, preferably in the order given.

Preferably, moreover, the first comparison index, the second comparison index and the third comparison index are obtained from the ratio respectively between the amplitude of each motor evoked potential MEP1, MEP2 and MEP3 and the amplitude of the control motor evoked potential MEPc.

Even more preferably, in the method, steps a1), a2) and a3) described above are repeated a predetermined number of times, so that in each of said steps a1), a2) and a3) a multiplicity of amplitudes of first MEP1, second MEP2 and third MEP3 motor evoked potentials are recorded. Moreover, in each of the steps b1), b2) and b3) the generation of said first, second and third comparison index respectively comprises:
for the first comparison index, the step of calculating the ratio between an average of the amplitudes of said multiplicity of first motor evoked potentials MEP1, and an average of the amplitudes of a multiplicity of control motor evoked potentials MEPc;
for the second comparison index, the step of calculating the ratio between an average of the amplitudes of said multiplicity of second motor evoked potentials MEP2, and an average of the amplitudes of a multiplicity of control motor evoked potentials MEPc;
for the third comparison index, the step of calculating the ratio between an average of the amplitudes of said multiplicity of third motor evoked potentials MEP3, and an average of the amplitudes of a multiplicity of control motor evoked potentials MEPc.

Preferably, the control motor evoked potential MEPc (represented by a dashed line in FIG. 1 and overlapped to the motor evoked potentials MEP1, MEP2 and MEP3 only for a virtual comparison) is recorded in the region of the body of the subject, preferably near the first dorsal interosseous muscle (FDI), and is obtained as a result of a transcranial magnetic stimulation with a control stimulus, generated by a magnetic stimulator, preferably having an intensity such as to evoke a motor evoked potential of about 1 mV at the level of such a region of the body of the subject (FDI).

Preferably, once the motor threshold at rest has been defined as the minimum intensity of the stimulus generated by the magnetic stimulator suitable to evoke a motor evoked potential of an amplitude equal to at least 50 μV (micro Volt) in the region of the body of the subject in at least 50% of cases of a series of stimulations, the first and/or second conditioning stimulus C1, C2 have an intensity lower than such a motor threshold at rest and preferably about 70% of the motor threshold at rest.

Preferably, the first predetermined time interval ISI1 is less than or equal to 3 ms, and preferably greater than or equal to 1 ms.

Moreover, preferably the second predetermined time interval ISI2 is less than or equal to 10 ms and greater than 3 ms, preferably greater than or equal to 7 ms.

In other words, a preferred embodiment variant of the invention provides for the setting of the magnetic stimulator so that it generates a magnetic impulse (control stimulus T) at an intensity required to evoke a motor evoked potential of about 1 mV at the level of the dorsal interosseous muscle. When the control stimulus T is preceded by a conditioning (magnetic) stimulus C1, C2 of different intensity, or by a conditioning (electric) stimulus C3, the amplitude of the recorded motor evoked potential is modulated in a positive or negative way (i.e. amplified or reduced), according to the time interval ISI elapsed between the conditioning stimulus C1, C2, C3 and the control stimulus T (as shown in FIG. 1, wherein according to the stimulation protocol the motor evoked potentials MEP1, MEP2 and MEP3 represented by a continuous line have an amplitude greater or less than the control motor evoked potential MEPc represented by a discontinuous line). This technique, defined as double impulse, allows evaluating the intracortical circuit inhibitors and facilitators.

In particular, the steps of the method from a1) to b1) allow evaluating the so-called short-interval intracortical inhibition (SICI), i.e. the intracortical circuit inhibitors may be investigated. From neuropharmacological studies, it is assumed that the SICI is dependent on the activity of the GABAA receptors (Kujirai T et al. Corticocortical inhibition in human motor cortex. J Physiol. 1993;471:501-519). As described above, the SICI is preferably evaluated by the administration of two stimuli, one conditioning stimulus C1, preferably 70% of the motor threshold at rest (defined as minimum intensity of the stimulator capable of evoking a motor evoked potential of at least 50 μV in 5 of 10 consecutive tests), and the control stimulus T. The time lapse between the two stimuli C1 and T varies preferably in a casual manner and includes the following interstimulus intervals (ISI1): 1, 2, 3 ms.

Preferably, a series of pairs of conditioning C1—control T stimuli is administered to the subject for each interval (such as 10 pairs of conditioning stimulus—control stimulus) and a series of control stimuli T in the absence of the conditioning stimulus (such as 14 control stimuli). The amplitude of the motor evoked potential MEP1 is recorded for each series of pairs of stimuli and is compared with the average of the amplitudes of the control motor evoked potentials MEPc of the series of control stimuli T acquired in the absence of conditioning stimulus.

An example of an embodiment provides for calculating the average of the amplitudes of a series of motor evoked potentials MEP1 (such as 10 motor evoked potentials) for each interstimulus interval (such as 1 ms, 2 ms e 3 ms) and dividing it by the average of the amplitudes of the series of control motor evoked potentials MEPc (such as 14) obtained with the only control stimulus T in the absence of the conditioning stimulus, therefore obtaining an average SICI ratio for each interstimulus interval ISI1.

Preferably, in a second step, the average of the above average SICI ratios obtained for each determined interstimulus interval (for example, for the SICI, the average of the average SICI ratios obtained for each predetermined interstimulus interval ISI1, for example the average of the ratios obtained at 1, 2 and 3 ms, is carried out) is then calculated. The average of the above average SICI ratios therefore represents the first comparison index (or average SICI index).

Preferably, the method further allows also evaluating the intracortical facilitation (ICF), which allows investigating the combined effect of the secondary facilitation to inhibitor and exciter mechanisms mediated by GABAA and NMDA receptors, respectively (Ziemann U et al. Interaction between intracortical inhibition and facilitation in human motor cortex. J Physiol. 1996;496:873-881). The intracortical facilitation is evaluated by following the steps of the method from a2) to b2) described above. In other words, said stimulation method used for the evaluation of the SICI is used but, this time, increasing the interstimulus interval ISI2 to larger values with respect to those used in the SICI (values such as 7, 10, and 15 ms) and preferably using the same number of stimuli used in the SICI.

An embodiment example provides for calculating the average of the amplitudes of a series of motor evoked potentials MEP2 (such as 10 motor evoked potentials) for each interstimulus interval ISI2 (such as 7 ms, 10 ms e 15 ms) and dividing it by the average of the amplitudes of the series of control motor evoked potentials MEPc (such as 14) obtained with the control stimulus T alone in the absence of the conditioning stimulus C2, therefore obtaining an average ICF ratio for each interstimulus interval ISI2.

Subsequently, as for the evaluation of the SICI, the average of the above average ICF ratios obtained for each determined interstimulus interval ISI2 (such as at 7, 10, and 15 ms) is then calculated. Such an average of the above average ICF ratios therefore represents the second comparison index (or average ICF index).

Two values are thus obtained: the average SICI index (preferably calculated as the average of the average SICI ratios at 1, 2, 3 ms) and the average ICF index (preferably calculated as the average of the average ICF ratios at 7, 10, 15 ms).

Another step of the method allows evaluating the short-latency afferent inhibition (SAI), so as to determine in a non-invasive manner the inhibitor circuits in the sensorimotor cortex (Tokimura H et al. Short latency inhibition of human hand motor cortex by somatosensory input from the hand. J Physiol. 2000;523:503-513). The SAI is therefore used to determine in vivo the cholinergic activity deficit. In this step, the conditioning stimulus C3 is no longer administered by the same coil for magnetic stimulation for which the control stimulus T is given, instead it is administered by an electrical stimulator positioned on a further region of the body of the subject, preferably on the arm, for example on the median nerve, near the wrist (right or left according to the stimulated area on the cranium).

Preferably, the electrical conditioning stimulus C3 for nervous electrical stimulation is 200 μs, preferably with the cathode proximally positioned (towards the elbow) at a distance of 4 cm from the anode.

Preferably, the stimulus is administered by a bar electrode.

Preferably, the intensity of the electrical stimulus is regulated so as to be suitable to evoke a slight movement at the level of the thumb of the right (or left) hand.

Also in this case, an embodiment example provides for calculating the average of the amplitudes of a series of motor evoked potentials MEP3 (such as 10 motor evoked potentials) for each interstimulus interval ISI3 (such as 20 ms and 24 ms) and dividing it by the average of the amplitudes of the series of control motor evoked potentials MEPc (such as 14) obtained only with the control stimulus T and in the absence of the conditioning stimulus C3, therefore obtaining an average SAI ratio for each interstimulus interval ISI3.

As for the evaluation of the SICI and of the ICF, the average of the above average SAI ratios obtained for each determined interstimulus interval (such as at 20 and 24 ms) is then calculated. Such an average of the above average SAI ratios therefore represents the average SAI index (third comparison index).

At the end of the procedure, the three comparison indices are therefore obtained: the average SICI index (1, 2, 3 ms), the average ICF index (7, 10, 15 ms) and the average SAI index (20, 24 ms).

Preferably, in a further step the diagnostic comparison index is therefore calculated, from the ratio:

(average SICI index)/(average ICF index)/(average SAI index).

In a particularly innovative and advantageous manner, the above diagnostic comparison index allows dividing the patients in two groups, since the patients with Alzheimer's dementia have values less than 0.98, while, for example, patients with frontotemporal disease have values greater than 0.98.

The method described so far is implemented by an electronic apparatus 1, also object of the present invention, suitably configured to allow the execution of the method and described below with particular reference to figures from 2 to 4. However, it is clear that the electronic apparatus 1 described below is not strictly bound to the exclusive use of carrying out the method described above, but may also be used to carry out other types of management and generation of electrical impulses. In particular, FIGS. 2 and 3 schematically show a stimulation management system according to a first embodiment variant, wherein FIG. 2 shows the configuration for the single magnetic stimulation (for example in the case of evaluating the SICI and the ICF), while FIG. 3 shows the configuration of the system for the magnetic stimulation preceded by an electrical conditioning stimulus (for example as in the case of evaluating the SAI).

FIG. 4 shows a system capable of simultaneously managing all of the configurations through an integrated device, shown below.

The electronic apparatus 1 according to the present invention is suitable to manage a stimulator 2 for transcranial magnetic stimulation, an electrical stimulator 3 for nervous electrical stimulation and an electromyographic signal or data acquisition device 4. The electronic apparatus 1 comprises:
a synchronization interface 10 suitable to be connected to the stimulator 2 for transcranial magnetic stimulation and to the electrical stimulator 3;
a first communication interface 11 suitable to communicate with a remote data processing and configuration device 12, such as a computer;
a second communication interface 13, suitable to communicate with an electromyographic signal detection and/or acquisition unit 4;
an impulse generation and synchronization unit 14 operatively connected with the synchronization interface 10, with the first 11 and second communication interface 13 and comprising a logical processing unit 141, such as a field programmable gate array (FPGA) based processor or unit.

The logical processing unit 141 is configured to generate a first trigger electrical impulse A suitable to be received by the stimulator 2 for transcranial magnetic stimulation and/or a second trigger electrical impulse A′ suitable to be received by an electrical stimulator 3 for nervous electrical stimulation through the synchronisation interface 10. The logical processing unit 141 is configured to generate, after a predetermined time interval, a third trigger electrical impulse B suitable to be received by the stimulator 2 for transcranial magnetic stimulation.

Preferably, the logical processing unit 141 is configured to generate a fourth synchronisation electrical impulse S suitable to be received by the electromyographic signal acquisition device 4.

In an embodiment variant, such a fourth synchronisation electrical impulse S is said third trigger electrical impulse B.

In an advantageous embodiment variant, for example shown in FIG. 4, the electronic apparatus 1 is an integrated device, i.e. a single device, further comprising an electronic processing, communication and control board 15 (preferably a single-board computer, for example of the Raspberry Pi type) comprising the first communication interface 11 suitable to communicate with the remote data processing and configuration device 12 (such as a computer), the second communication interface 13 suitable to communicate with the electromyographic signal acquisition and/or detection unit 4 and a communication and configuration interface 151 operatively connected to the impulse generation and synchronisation unit 14. Furthermore, such an electronic processing, communication and control board 15 comprises at least one processor 152 and storage means, wherein the processor is configured to transmit signals or data relative to the configuration parameters of the logical processing unit 141 stored in the storage means, to the impulse generation and synchronization unit 14.

In the embodiment variant wherein the electronic apparatus 1 is an integrated device (FIG. 4), preferably the remote data processing and configuration device 12 is configured only to carry out the initial configuration of the electronic processing communication and control board 15 and optionally the impulse generation and synchronization unit 14 through the communication and configuration interface 151 with the electronic processing communication and control board 15. Preferably, in this variant, once the configuration of the electronic processing communication and control board 15 and the impulse generation and synchronisation unit 14 has been carried out, the electronic apparatus 1 is suitable to autonomously (without the need to be connected to the remote data processing and configuration device 12) carry out the tasks for which it was configured. This happens by virtue of the fact that the processor 152 is configured to perform all the processes described in the method according to the present invention, suitable to obtain the three comparison indices: the average SICI index, the average ICF index, the average SAI index and the diagnostic comparison index. Preferably, the output of the results of such processes is provided by printing the diagnosis index through a printer 17 or other on-screen display device.

Moreover, preferably the electronic apparatus 1 comprises an electronic level adjustment device 16 suitable to raise or lower the voltage levels of the trigger and synchronisation electrical impulses A, A′, B, S to adapt them to the voltage values receivable by each of said stimulator 2 for transcranial magnetic stimulation, electrical stimulator 3 for nervous electrical stimulation and electromyographic signal or data acquisition device 4.

It is also clear that the object of the present invention is a stimulation management system 100 comprising an electronic apparatus 1 described in the previous paragraphs and at least one stimulator 2 for transcranial magnetic stimulation, one electrical stimulator 3 for nervous electrical stimulation and one electromyographic signal or data acquisition device 4, connected to such an electronic apparatus.

With reference to FIG. 2, the operation of the stimulation management system is described in detail below. The stimulator 2 for transcranial magnetic stimulation (such as a Magstim BiStim2 system, Magstim Company, Oxford, UK) generates the conditioning stimulus and the control stimulus at the rising edge of the trigger impulses A and B coming from the impulse generation and synchronisation unit 14 (such as a BIOPAC Systems UIM100C device). Such conditioning and control stimuli are delivered by the coil 20 by magnetic stimulation on the cranium of the subject. Preferably, the amplitude of the trigger impulses A and B is 5 V. Moreover, the amplitude of the magnetic impulses is manually regulated by a roller on the panel of the stimulator, selecting it through a suitable protocol which considers the amplitude of the acquired motor evoked potential.

Preferably, the trigger impulses A and B are 20 ms.

Preferably, the user software on the computer manages both the configuration phase of the trigger impulses, and the acquisition and analysis of data received (e.g. motor evoked potentials).

FIG. 3 shows the configuration of the stimulation management system 100, when the electrical stimulator for nervous stimulation is provided (such as during the evaluation of the SAI). In this configuration, the electrical stimulator 3 for electrical nervous stimulation (such as a BIOPAC STMISOLA, BIOPAC Systems Inc.) generates the electrical conditioning stimulus as a function of the trigger impulse A′ coming from the impulse generation and synchronisation unit 14 (the BIOPAC Systems UIM100C device). This electrical conditioning stimulus is dispensed by means of the bar electrode 30 applied on the arm of the subject. The trigger impulse (trigger) A′ is configured to determine both the duration of the electrical conditioning stimulus (which will be equal to the length of the trigger impulse A′) and the amplitude of the stimulus (which, for example, will be equal to about 20 times the amplitude of the trigger impulse A′).

Preferably, the trigger impulses A′ and B are 200 μs and 20 ms.

FIG. 4 shows the configuration of the stimulation management system 100, in which the electrical apparatus is an integrated device as described above.

In this variant, the electronic processing, communication and control board 15 (the single-board computer) is configured to manage the acquisition of electromyographic data, the Ethernet/Wifi communication with the remote device (computer) 12, the USB/Wifi communication with any printer 17 and the interaction with the user through a user interface (for example, switches, buttons, LEDs and 7 segment display).

In addition, the electronic processing, communication and control board 15 is configured to send the configuration signals to the pulse generation and synchronization unit 14 as a function of the stimulation protocol (pulse number, sequence, temporal distance . . . ); for example, the configuration signals comprise a specific configuration of the logical processing unit based on FPGA suitable for creating specific trigger and synchronization signals for a particular stimulation protocol).

In addition, preferably, the electronic processing, communication and control board 15 is configured to process EMG signals and generate the results of processing.

In this embodiment variant, the pulse generation and synchronization unit 14 is a FPGA-based board (such as a Terasic DE0 board). This unit is configured to generate the trigger impulses A, A′, B and synchronization S properly timed and synchronized. In addition, preferably the FPGA board manages the user interface at a low level. In other words, preferably the user interface (switches, buttons, LEDs and 7-segment display) is physically connected to the FPGA board that is configured to read and/or write the physical values (voltages). So, for example, to send a message to the user via the display, the electronic processing, communication and control board 15 (for example, the single board computer) sends the message to the FPGA board, which in turn configures and sends the physical values to the display to bring up the message.

The effectiveness of the method and apparatus according to the present invention were also evaluated by a study of 81 patients who met the current clinical criteria for Alzheimer's disease (AD) (Dubois et al., Advancing research diagnostic criteria for Alzheimer's disease: the IWG-2 criteria. The Lancet Neurology 2014; 13: 614-629) and of 61 patients who met the current clinical criteria for frontotemporal disease (FTD) (Gorno-Tempini et al. Classification of primary progressive aphasia and its variants. Neurology 2011; 76: 1006-1014; Rascovsky et al. Sensitivity of revised diagnostic criteria for the behavioural variant of frontotemporal dementia. Brain 2011; 134: 2456-2477), recruited consecutively by the Center for brain aging and neurodegenerative diseases of the Department of Clinical and Experimental Sciences of the University of Brescia and the Fondazione Santa Lucia, Rome, Italy. Also 32 healthy subjects of the same age were recruited from spouses and healthy volunteers as a control group (HC).

The diagnostic evaluation involved a review of the complete medical history, a semi-structured neurological examination, an assessment of the full state of mind for standardized neuropsychological evaluation, and a brain MRI scan.

All subjects were drug naive or had stopped for weeks medical treatment prior to the study participation.

Full written informed consent was obtained from all subjects on the basis of the Declaration of Helsinki. The study protocol was approved by a Research Ethic Committee in each center.

In this case, the transcranial magnetic stimulation was performed with an “eight-shaped” coil (with each circle of the “eight” shape having a 70 mm diameter) connected to a Magstim Bistim2 system (Magstim Company, Oxford, UK). The magnetic stimuli had a single-phase current waveform (100 ms rise time, decay time at zero over 800 ms). The motor evoked potentials were recorded from the first dorsal interosseous muscle (FDI) of the right hand through Ag/AgCl surface electrodes placed in a “belly-tendond” arrangement and acquired using an EMG Biopac MP-150 (BIOPAC Systems Inc., Santa Barbara, Calif., USA), or an amplifier Digitimer D360 (Digitimer, UK). The EMG signals were filtered with a band pass filter (10 Hz-1 kHz), sampled (sampling frequency: 5 kHz) and stored on a computer for off-line analysis.

The transcranial magnetic stimulation coil was held tangentially on the region of the scalp corresponding to the primary motor of the hand contralateral to the target muscle, with the coil handle indicating 45° posteriorly and laterally to the sagittal plane. The motor hot spot was defined as the position in which the magnetic stimulation consistently produced the largest motor evoked potential, when stimulated at 120% of motor resting threshold (RMT) of the target muscle and was marked with a marker on the scalp to ensure the constant positioning of the coil throughout the experiment.

The motor threshold at rest RMT was defined as the minimum intensity of the stimulus necessary to produce motor evoked potentials with an amplitude of at least 50 mV in 5 out of 10 consecutive repetitions in a complete muscle relaxation.

SICI and ICF were evaluated at rest through a paired-pulse paradigm, as previously reported (Kujirai T et al. Corticocortical inhibition in human motor cortex. J Physiol. 1993;471:501-519; Ziemann U et al., Interaction between intracortical inhibition and facilitation in human motor cortex. J Physiol 1996; 496: 873-881). In summary, the conditioning stimulus (CS) was set at an intensity equal to 70% RMT, while the control stimulus (TS) was adjusted to evoke a motor evoked potential of approximately 1 mV peak-to-peak in the first dorsal interosseous muscle relaxed. Several interstimulus intervals (ISIS) between the CS and TS were employed to preferentially study both SICI (1, 2, 3, 5 ms) and ICF (7, 10, 15 ms) (Kujirai T et al. Corticocortical inhibition in human motor cortex. J Physiol. 1993;471:501-519; Ziemann U et al., Interaction between intracortical inhibition and facilitation in human motor cortex. J Physiol 1996; 496: 873-881).

The SAI was studied using a technique previously described (Tokimura et al., Short latency inhibition of human hand motor cortex by somatosensory input from the hand. J Physiol 2000; 523 Pt 2: 503-513). The control stimuli were a single pulse (200 mS) of electrical stimulation applied through bipolar electrodes to the right median nerve at the wrist (proximal cathode). The intensity of the conditioning stimulus was set to a value slightly higher than the driving threshold suited to evoke a visible contraction of the thenar muscles, while the control stimulus was adjusted to evoke a motor of about 1 mV peak-to-peak potential. The conditioning stimulus to the peripheral nerve has preceded the control stimulus to different ISI (−4, 0, +4, +8 ms). Each ISI was determined with respect to the latency of the N20 component of the somatosensory evoked potential induced by stimulation of the right median nerve.

Ten stimuli were delivered for each ISI for all stimulation paradigms (SICI, ICF and SAI) and fourteen evoked potential monitoring were recorded in response to the control stimuli only, for each paradigm, to all participants in a pseudo randomized sequence. The amplitude of evoked potentials of conditioning was expressed in relation to the average of the responses evoked unconditional (control evoked potentials). The interval between a test and the other was set at 5 sec (±10%).

Throughout the experiment, it was monitored complete muscle relaxation using an audio-visual feedback where appropriate. If the quality of the data was found to be degraded by patient motion, the protocol was again repeated, and the initial data discarded. The tests were discarded if the electromyographic activity exceeded 100 microvolts in 250 ms prior to the dispensing of the magnetic stimulation stimulus. All patients were able to understand the instructions and obtain the complete muscle relaxation.

All patients AD and FTD, as well as all the HC, were evaluated both for SICI-ICF and for SAI.

The comparison of the clinical characteristics was carried out using a one-way ANOVA test or Fisher-Freeman-Halton exact test, as appropriate.

The neurophysiological parameters were compared by repeated measures ANOVA with ISI (1, 2, 3, 5, 7, 10, 15 ms for SICI-ICF; −4, 0, +4, +8 ms for SAI) as an intra-subject factor and GROUP (HC vs FTD vs AD) as between-subjects factor. When a significant main effect was achieved, post hoc tests were conducted with Bonferroni correction for multiple comparisons in order to analyze differences between the groups to the respective ISI. Mauchly test was used to test hypotheses of sphericity, while the Greenhouse-Geisser epsilon determination was used for the correction in the event of violation of sphericity.

The analysis of the ROC curve (Receiver Operating Characteristics) (FIG. 6) was used to determine the area under the curve (AUC) values comprising 95% confidence interval (CI) and cut-off points were established to obtain high levels of sensitivity and specificity.

Statistical significance was assumed at a p-value smaller than 0.05.

### Results

Eighty-one AD patients (age 71.2±8.1), sixty-one FTD (age 65.6±9.2), and thirty-two HC (age 61.5±10.3) were included in the study.

The repeated measures ANOVA performed on SICI-ICF revealed a significant interaction ISIxGROUP, F (6.45, 551.76)=24.33, p<0.001, η2 partial=0.22, ϵ=0.54. The post-hoc comparisons revealed a significant difference between AD and FTD at ISI 1, 2, 7, 10, 15 ms (all p-values <0.001), a significant difference between FTD and HC at ISI 2, 3, 7, 10, 15 ms (all p-values <0.001) and significant differences between AD and HC in all ISI (see FIG. 5).

For the SAI paradigm, the repeated measures ANOVA revealed a significant interaction ISIxGROUP, F (4.69, 400.57)=8.85, p<0.001, η2 partial=0.09, ϵ=0.78. The post hoc comparisons revealed a significant difference between FTD and HC for ISI values 0 and +4 ms (all p values <0.001). No significant differences were observed between FTD and HC (see FIG. 5).

A ROC curve (FIG. 6) was traced to compare the efficacy of the neurophysiological measures in the differentiation of patients with AD from those with FTD. The average SICI-ICF (1, 2, 3, 7, 10, 15 ms) revealed an AUC of 0.86 (p<0.001, 95% confidence interval 0.80 to 0.92), and the average SAI (0, +4 ms) revealed an AUC of 0.90 (p<0.001, 95% confidence interval 0.84 to 0.95). The combination of multiple measurements, in particular the SICI/ICF/SAI report revealed an AUC of 0.93 (p<0.001, 95% confidence interval 0.90 to 0.97) (see FIG. 5). At the best score cut-off of 0.98, sensitivity was 91.8%, specificity 88.6% and accuracy 90.0%.

Innovatively, the present invention allows calculating a diagnostic index that allows on the one hand to facilitate early diagnosis of Alzheimer's disease in a non-invasive manner and on the other hand, it also helps improve the differential diagnosis with respect to other dementia pathologies (frontotemporal disease).

In addition, in an innovative way the apparatus according to the present invention allows obtaining in a non-invasive manner an early and differential diagnostic index of the disease, also making it feasible in most diagnostic centers.

Additionally, advantageously, the electronic apparatus allows both obtaining the correct timing and synchronization of the magnetic and/or electrical stimuli with the recording of electromyographic signals, and facilitating the automation of the diagnostic index generation method described.

Advantageously, moreover, the same electronic apparatus, especially in its integrated configuration, facilitates the application of the method according to the present invention in clinical practice, also due to the relative rapidity of obtaining the result and to the relative reduction of costs compared to the prior art techniques that use radiolabeled tracers.

In particular, the electronic apparatus in the integrated configuration is suitable to be interfaced directly with the magnetic stimulator, eliminating the need of having to program complex electronic instruments and providing an immediate result (such as the diagnostic index) (the method execution time according to the present invention may also be less than 10 minutes) and easy to interpret.

It is clear that a man skilled in the art may make changes to the invention described above in order to meet incidental needs, all falling within the scope of protection defined in the following claims.

1. Method of generating a diagnostic index comprising the steps of:
a1) stimulating a subject using transcranial magnetic stimulation with at least one first conditioning stimulus and, after a first predetermined time interval, further stimulating the subject using transcranial magnetic stimulation with a control stimulus and recording amplitude of a first motor evoked potential generated by a region of the body of the subject, near the first dorsal interosseous muscle, as a result of said conditioning and control stimuli;
b1) generating a first comparison index by comparing said amplitude of the first motor evoked potential and amplitude of a control motor evoked potential;
a2) stimulating a subject using transcranial magnetic stimulation with at least a second conditioning stimulus and, after a second time interval greater than said first predetermined time interval, further stimulating the subject using transcranial magnetic stimulation with a control stimulus and recording amplitude of a second motor evoked potential generated by the region of the body of the subject as a result of said second conditioning and control stimuli;
b2) generating a second comparison index by comparing said amplitude of the second motor evoked potential and the amplitude of the control motor evoked potential;
a3) stimulating a subject using nervous electrical stimulation of a nerve associated with said region of the body with at least one electrical conditioning stimulus and, after a third predetermined time interval, further stimulating the subject using transcranial magnetic stimulation with a control stimulus and recording amplitude of a third motor evoked potential generated by the region of the body of the subject as a result of said third conditioning and control stimuli;
b3) generating a third comparison index by comparing said amplitude of the third motor evoked potential and the amplitude of the control motor evoked potential;
c) elaborating said first comparison index, said second comparison index and said third comparison index to obtain a diagnostic comparison index as a function of said first comparison index, said second comparison index and said third comparison index.

2. Method according to claim 1, wherein the diagnostic comparison index is a ratio between said first comparison index, said second comparison index and said third comparison index, in the order given.

3. Method according to claim 1, wherein the first comparison index, the second comparison index and the third comparison index are obtained from a ratio respectively between the amplitude of said first motor evoked potential and the amplitude of the control motor evoked potential, between the amplitudes of said second motor evoked potential and the amplitude of the control motor evoked potential and between the amplitude of said third motor evoked potential and the amplitude of the control motor evoked potential.

4. Method according to claim 1, wherein the steps a1), a2) and a3) are repeated a predetermined number of times, so that in each of said steps a1), a2) and a3) a multiplicity of amplitudes of first, second and third motor evoked potentials are recorded and wherein in each of the steps b1), b2) and b3) the generation of said first second and third comparison indices respectively comprises:
for the first comparison index, the step of calculating a ratio between an average of the amplitudes of said multiplicity of first motor evoked potentials, and an average of the amplitudes of a multiplicity of control motor evoked potentials;
for the second index, the step of calculating a ratio between an average of the amplitudes of said multiplicity of second motor evoked potentials, and an average of the amplitudes of a multiplicity of control motor evoked potentials;
or the third index, the step of calculating a ratio between an average of the amplitudes of said multiplicity of third motor evoked potentials, and an average of the amplitudes of a multiplicity of control motor evoked potentials.

5. Method according to claim 1, wherein the control motor evoked potential is recorded in said region of the body of the subject, near the first dorsal interosseous muscle, and is obtained as a result of a transcranial magnetic stimulation with a control stimulus, without any conditioning stimulus.

6. Method according to claim 5, wherein the control stimulus is generated by a magnetic stimulator and has an intensity to evoke a motor evoked potential of about 1 mV at a level of said region of the body of the subject.

7. Method according to claim 1, wherein said first and/or second conditioning stimuli are generated by a magnetic stimulator and wherein, having defined a motor threshold at rest as a minimum intensity of the stimulus generated by the magnetic stimulator to evoke a motor evoked potential of an amplitude equal to at least 50 mV in said region of the body of the subject in at least 50% of cases of a series of stimulations, said first and/or second conditioning stimuli have an intensity lower than said motor threshold at rest.

8. Method according to claim 7, wherein the intensity of said first and/or second conditioning stimuli is about equal to 70% of the motor threshold at rest.

9. Method according to claim 1, wherein said first predetermined time interval is less than or equal to 3 ms.

10. Method according to claim 1, wherein said second predetermined time interval is less than or equal to 10 ms and greater than 3 ms.

11. Electronic apparatus for the management of a stimulator for transcranial magnetic stimulation, of an electrical stimulator for nervous electrical stimulation and of an electromyographic signal or data acquisition device, said electronic apparatus being configured to allow execution of the method according to claim 1 and comprising:
a synchronization interface connected to the stimulator for transcranial magnetic stimulation and to the electrical stimulator;
a first communication interface to communicate with a remote data processing and configuration device;
a second communication interface to communicate with an electromyographic signal detection unit and/or acquisition unit;
an impulse generation and synchronization unit operatively connected with the synchronization interface, with the first and second communication interfaces and comprising a logical processing unit,
said logical processing unit being configured to generate:
a first synchronization electrical impulse to be received by the stimulator for transcranial magnetic stimulation; and/or
a second synchronization electrical impulse to be received by an electrical stimulator for nervous electrical stimulation through the synchronization interface and, after a predetermined time interval, said logical processing unit being configured to generate a third synchronization electrical impulse to be received by the stimulator for transcranial magnetic stimulation.

12. Electronic apparatus according to claim 11, wherein the logical processing unit is configured to generate a fourth synchronization electrical impulse to be received by the electromyographic signal acquisition device.

13. Electronic apparatus according to claim 12, wherein said fourth synchronization electrical impulse is said third synchronization electrical impulse (B).

14. Electronic apparatus according to claim 11, comprising
an electronic processing, communication and control board comprising the first communication interface to communicate with the remote data processing and configuration device, the second communication interface to communicate with the electromyographic signal detection unit and/or acquisition unit of, a communication and configuration interface operatively connected to the impulse generation and synchronization unit,
at least one processor and storage, said processor being configured to transmit signals or data relative to the configuration parameters of the logical processing unit stored in the storage, to the impulse generation and synchronization unit.

15. Electronic apparatus according to claim 14, wherein the processor is configured to elaborate said first comparison index, said second comparison index and said third comparison index to obtain a diagnostic comparison index as a function of said first comparison index, said second comparison index and said third comparison index.

16. Electronic apparatus according to claim 11, further comprising an electronic level adjustment device to raise or lower voltage levels of the synchronisation electrical impulses to adapt the voltage levels to voltage values receivable by each of said stimulator for transcranial magnetic stimulation, electrical stimulator for nervous electrical stimulation and electromyographic signal or data acquisition device.

17. Electronic apparatus according to claim 16, wherein said electronic apparatus is a single integrated device comprising the electronic processing, communication and control board, the impulse generation and synchronization unit and the electronic level adjustment device.

18. Electronic apparatus according to claim 11, wherein the logical processing unit is a Field Programmable Gate Array based device.

19. Electronic apparatus according to claim 11, wherein the electronic processing, communication and control board is a single-board computer.

20. Electronic apparatus according to claim 14, wherein said electronic apparatus is an integrated device, directly connectable to the stimulator for transcranial magnetic stimulation and to the electrical stimulator.

21. Stimulation management system comprising an electronic apparatus according to claim 11 and at least a stimulator for transcranial magnetic stimulation, an electrical stimulator for nervous electrical stimulation and an electromyographic signal or data acquisition device, connected to the electronic apparatus.
